SEQUENCE LISTING (52 sequences)

SEQ ID NO: 1 - DNA, 20 bp, Artificial Sequence
  Primer
tgctcaagcaaatattgcac

SEQ ID NO: 2 - DNA, 23 bp, Artificial Sequence
  Primer
gtttgggatccaattaaccctct

SEQ ID NO: 3 - DNA, 20 bp, Artificial Sequence
  Primer
actgcagcccttcaactacg

SEQ ID NO: 4 - DNA, 20 bp, Artificial Sequence
  Primer
aacgcctacctcatgtttgg

SEQ ID NO: 5 - DNA, 24 bp, Artificial Sequence
  Primer
gtaggtgcacttgatgtgctttac

SEQ ID NO: 6 - DNA, 25 bp, Artificial Sequence
  Primer
aacaccatattgagtctttcaaagc

SEQ ID NO: 7 - DNA, 25 bp, Artificial Sequence
  Primer
ctaagctcaccaagaggaagttgat

SEQ ID NO: 8 - DNA, 20 bp, Artificial Sequence
  Primer
gcttctgggtctcctgctca

SEQ ID NO: 9 - DNA, 23 bp, Artificial Sequence
  Primer
ggtcaagatttgataaagtggga

SEQ ID NO: 10 - DNA, 21 bp, Artificial Sequence
  Primer
gcactccacttgtgcgttttc

SEQ ID NO: 11 - DNA, 20 bp, Artificial Sequence
  Primer
tccgtgttgagtatggacga

SEQ ID NO: 12 - DNA, 19 bp, Artificial Sequence
  Primer
tgtttttggtggagcgatg

SEQ ID NO: 13 - DNA, 19 bp, Artificial Sequence
  Primer
agtcggctccccttttctc

SEQ ID NO: 14 - DNA, 20 bp, Artificial Sequence
  Primer
gcgatctgacctcatttttg

SEQ ID NO: 15 - DNA, 20 bp, Artificial Sequence
  Primer
ccaggtaagcagctcagtgt

SEQ ID NO: 16 - DNA, 20 bp, Artificial Sequence
  Primer
acctcccatttgtgtcttgc

SEQ ID NO: 17 - DNA, 21 bp, Artificial Sequence
  Primer
cgaaagatggacttccatgac

SEQ ID NO: 18 - DNA, 22 bp, Artificial Sequence
  Primer
caagttgagattcttcctgcaa

SEQ ID NO: 19 - DNA, 20 bp, Artificial Sequence
  Primer
ccaagcacgaatgaactcaa

SEQ ID NO: 20 - DNA, 20 bp, Artificial Sequence
  Primer
gcgggttctcgttgtacact

SEQ ID NO: 21 - DNA, 20 bp, Artificial Sequence
  Primer
ctctgtgtcggctttctggt

SEQ ID NO: 22 - DNA, 20 bp, Artificial Sequence
  Primer
ataaacctcacgccacacct

SEQ ID NO: 23 - DNA, 20 bp, Artificial Sequence
  Primer
gcccttcttcttttctgcaa

SEQ ID NO: 24 - DNA, 20 bp, Artificial Sequence
  Primer
agtgccacctcaacaaatcc

SEQ ID NO: 25 - DNA, 20 bp, Artificial Sequence
  Primer
caccctcttcaccacctcat

SEQ ID NO: 26 - DNA, 20 bp, Artificial Sequence
  Primer
catcccctgttggagttttc

SEQ ID NO: 27 - DNA, 20 bp, Artificial Sequence
  Primer
gttcacaggctgttgggttt

SEQ ID NO: 28 - DNA, 20 bp, Artificial Sequence
  Primer
cgtgttgctcatgtgttgtg

SEQ ID NO: 29 - DNA, 20 bp, Artificial Sequence
  Primer
gttttgcgagctcattgtca

SEQ ID NO: 30 - DNA, 21 bp, Artificial Sequence
  Primer
tgtgtgtggtctgtaggcact

SEQ ID NO: 31 - DNA, 24 bp, Artificial Sequence
  Primer
tgtatgtacacacggagagagaga

SEQ ID NO: 32 - DNA, 20 bp, Artificial Sequence
  Primer
tgaggggaagaggtttgatg

SEQ ID NO: 33 - DNA, 22 bp, Artificial Sequence
  Primer
ggagccaccatgaaattataca

SEQ ID NO: 34 - DNA, 20 bp, Artificial Sequence
  Primer
cacttattgcgattggttca

SEQ ID NO: 35 - DNA, 19 bp, Artificial Sequence
  Primer
ctaccccatcgcttgacac

SEQ ID NO: 36 - DNA, 20 bp, Artificial Sequence
  Primer
ggagacttgtttgccacaga

SEQ ID NO: 37 - DNA, 22 bp, Artificial Sequence
  Primer
ctcacctttaccccttcatttt

SEQ ID NO: 38 - DNA, 25 bp, Artificial Sequence
  Primer
gtttcctcatcttctgagaaccatc

SEQ ID NO: 39 - DNA, 24 bp, Artificial Sequence
  Primer
ggtctccttcgctaacataaccaa

SEQ ID NO: 40 - DNA, 20 bp, Artificial Sequence
  Primer
gttgccctcgagttgtagta

SEQ ID NO: 41 - DNA, 19 bp, Artificial Sequence
  Primer
acacacacacacacacgaa

SEQ ID NO: 42 - DNA, 18 bp, Artificial Sequence
  Primer
gcaccaagcagaacaatc

SEQ ID NO: 43 - DNA, 21 bp, Artificial Sequence
  Primer
cttacagataaatggctcaaa

SEQ ID NO: 44 - DNA, 18 bp, Artificial Sequence
  Primer
aagcaagaaagggatggt

SEQ ID NO: 45 - DNA, 18 bp, Artificial Sequence
  Primer
aggcatcagttcatccaa

SEQ ID NO: 46 - DNA, 21 bp, Artificial Sequence
  Primer
ggaaggtgagagaaatcaagt

SEQ ID NO: 47 - DNA, 22 bp, Artificial Sequence
  Primer
ttattccaccaaagtctacctc

SEQ ID NO: 48 - DNA, 21 bp, Artificial Sequence
  Primer
tcctcaccaatcacactattt

SEQ ID NO: 49 - DNA, 18 bp, Artificial Sequence
  Primer
aaggcggcactcgctcac

SEQ ID NO: 50 - DNA, 21 bp, Artificial Sequence
  Primer
gaacaactgaagacagcaaag

SEQ ID NO: 51 - DNA, 21 bp, Artificial Sequence
  Primer
ctggcttcaaatcaatcatac

SEQ ID NO: 52 - DNA, 21 bp, Artificial Sequence
  Primer
gaggagaggagagagaaagag

(19) United States
(12) Plant Patent Application Publication
(10) Pub. No.: US 2009/0265820 P1
(43) Pub. Date: Oct. 22, 2009

(54) Corylus plant named 'Red Dragon'

(50) ;

(76) Inventors: Shawn A. Mehlenbacher, Corvallis, OR (US); David C. Smith, Corvallis, OR (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/148,080

(22) Filed: Apr. 16, 2008

(51) Int. Cl. A01H 5/00

(52) U.S. Cl. PLT/152

(57) ABSTRACT

A new and distinct cultivar of Corylus plant named ‘Red Dragon,’ characterized by its outwardly spreading plant habit, twisting stems, rich dark burgundy-colored leaves, burgundy color of the catkins and leaf buds, and resistance to eastern filbert blight caused by the fungus Anisogramma anomala (Peck) E. Muller.

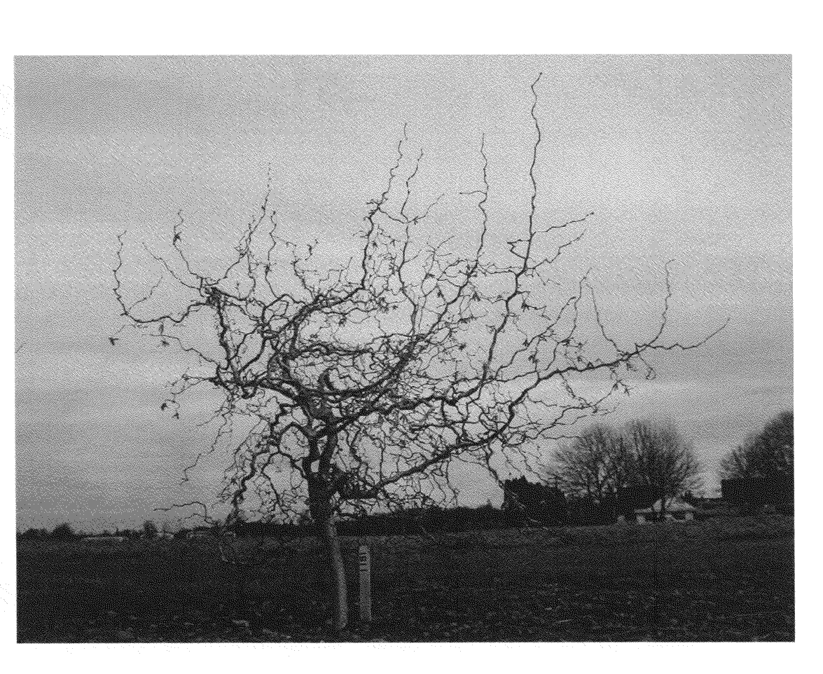

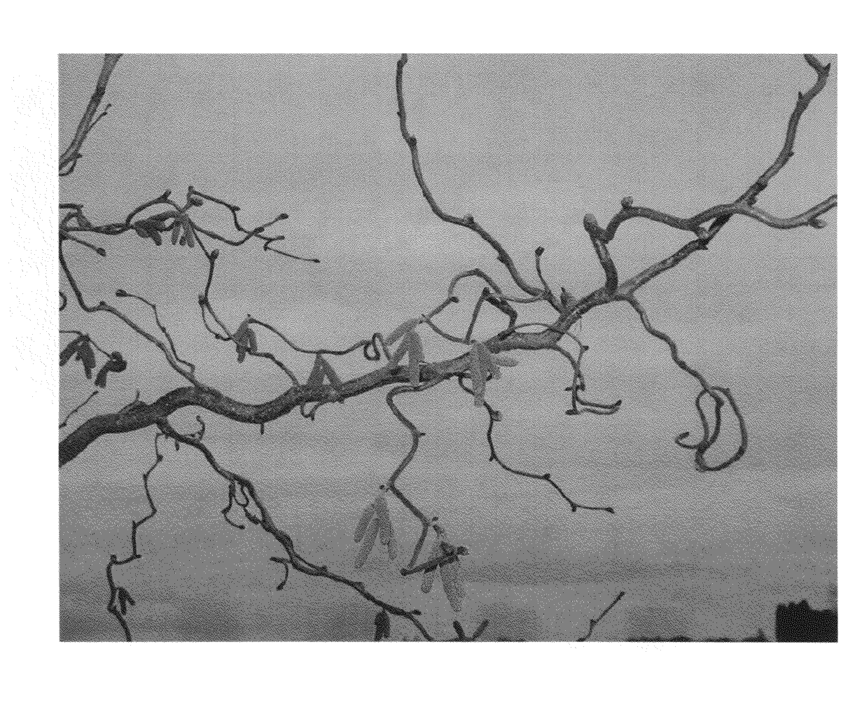

### LATIN NAME OF THE GENUS AND SPECIES OF THE PLANT CLAIMED

Corylus avellana

### VARIETY DENOMINATION

‘Red Dragon’

### BACKGROUND OF THE INVENTION

The present invention relates to a new and distinct cultivar of Corylus plant (hazelnut, filbert), botanically known as Corylus avellana, and hereinafter referred to by the name ‘Red Dragon.’

The new Corylus resulted from a controlled cross of female parent OSU 487.055 and male parent OSU 367.039 made in 1997 by Shawn A. Mehlenbacher and David C. Smith. Neither parent was protected by a plant patent. Hybrid seeds from the cross were harvested in August 1997, stratified, and seedlings grown in the greenhouse during the summer of 1998. From this cross, 42 seedlings with contorted growth habit were planted in the field in October 1998. ‘Red Dragon’ was discovered and selected by the inventors as a single plant within the progeny of the stated cross-pollination in a controlled environment in Corvallis, Oreg., USA. It was originally assigned the designation OSU 897.078, which indicates the row and tree location of the original seedling.

The female parent OSU 487.055 is from a cross of ‘Contorta’×VR 6-28 (which is a cross of ‘Riccia di Talanico’×‘Gasaway’), and the male parent OSU 367.039 is a red-leaf selection from open-pollination of ‘Contorta.’ The presumed pollen parent of OSU 367.039 is the red leaf cultivar ‘Rode Zeller’ (syn. ‘Rote Zellernuss’), which has a dominant allele at the leaf anthocyanin locus. A tree of ‘Rode Zeller’ was near the ‘Contorta’ tree from which open-pollinated nuts were collected. ‘Red Dragon’ and OSU 487.055 carry in heterozygous state a dominant allele for complete resistance to eastern filbert blight (EFB) from ‘Gasaway.’ Contorted growth habit is conferred by a recessive allele from ‘Contorta’ (syn. Corylus avellana var. contorta).

The new cultivar was asexually reproduced by rooted suckers (tie-off layerage of the suckers) annually for three years (2003-2005) and harvested in late November to early January. The layers of ‘Red Dragon’ were moderately vigorous, and rooted with a higher frequency and produced more roots than most other contorted selections. Asexual reproduction was also performed by whip grafting in late spring 2004. ‘Red Dragon’ is also suitable for propagation by micropropagation. The unique features of this new Corylus are stable and reproduced true-to-type in successive generations of asexual reproduction.

### SUMMARY OF THE INVENTION

The following traits have been observed and are determined to be the unique characteristics of ‘Red Dragon.’ These characteristics in combination distinguish ‘Red Dragon’ as a new and distinct cultivar:

1. Outwardly spreading plant habit.

2. Twisting stems.

3. Rich dark burgundy-colored developing leaves and rich dark burgundy-colored fully expanded leaves during the spring and summer.

4. Burgundy color of the catkins and leaf buds.

5. Resistance to eastern filbert blight caused by the fungus Anisogramma anomala (Peck) E. Muller.

6. Presence of random amplified polymorphic DNA markers 152-800 and 268-580 in DNA of ‘Red Dragon’ amplified by polymerase chain reaction (PCR). These two markers are linked to a dominant allele for resistance to eastern filbert blight from the cultivar ‘Gasaway,’ not patented.

7. Expression of incompatibility alleles S₆ and S₂₆ in the styles.

8. DNA fingerprints at 20 of 28 microsatellite marker loci differ from two plants of ‘Red Majestic’ (U.S. Plant Pat. 16,048).

In side-by-side comparisons conducted in Corvallis, Oreg., plants of the new Corylus differed from plants of the Corylus avellena cultivar ‘Contorta,’ not patented, and other cultivars and selections of Corylus avellena known to the inventors primarily in leaf coloration and plant size as plants of the cultivar ‘Contorta’ and other cultivars and selections of Corylus avellana had green-colored leaves and were larger than plants of the new Corylus. ‘Red Dragon’ leaves retain their dark red color better than ‘Red Majestic’ (U.S. Plant Pat. 16,048). The DNA fingerprint of ‘Red Dragon’ differs from that of two plants of ‘Red Majestic.’

### BRIEF DESCRIPTION OF THE PHOTOGRAPHS

The accompanying colored photographs illustrate the overall appearance of the new cultivar, showing the colors as true as it is reasonably possible to obtain in colored reproductions of this type.

The photograph on the first sheet comprises a side perspective view of a typical plant of ‘Red Dragon’ in Corvallis, Oreg.

The photograph on the second sheet is a close-up view of a typical plant of ‘Red Dragon.’

### DETAILED DESCRIPTION OF THE INVENTION

The cultivar ‘Red Dragon’ has not been observed under all possible environmental conditions. The phenotype may vary somewhat with variations in environment such as temperature and light intensity, without, however, any variance in genotype. The aforementioned photographs and following observations and measurements describe plants grown in Corvallis, Oreg. under commercial practice outdoors in the field during the fall, winter, and spring. Plants used for the photographs and description were about four years old. In the following description, color references are made to The Royal Horticultural Society Colour Chart, 1966 Edition, except where general terms of ordinary dictionary significance are used.
Botanical classification: Corylus avellana cultivar ‘Red Dragon.’
Parentage:
   Female, or seed, parent.—Corylus avellana selection OSU 487.055, not patented.
   Male, or pollen, parent.—Corylus avellana selection OSU 367.039, not patented.
Propagation:
   Type.—rooted suckers.
   Time to initiate roots.—About 30 days at 20° C.
   Time to produce a rooted young plant.—About six months at 22° C.
   Root description.—Fine to thick; freely branching; creamy white in color.
   Type.—whip grafting.
   Time to budbreak on the scions.—About 14 days at 25° C.
   Time to produce a grafted plant.—About six months at 25° C.
Plant description:
   General appearance.—Perennial shrub. Outwardly spreading plant habit.
   Growth and branching habit.—Freely branching; about 15 lateral branches develop per plant. Pinching, i.e., removal of the terminal apices, enhances branching with lateral branches potentially forming at every node. Strong and moderately vigorous growth habit. Stems twisting or “contorted.”
   Plant height.—About 2 meters.
   Plant diameter or spread.—About 2 meters.
   Lateral branch description.—Length: About 15 cm. Diameter: About 5 mm. Internode length: About 1.3 cm. Texture: Smooth, glabrous. Strength: Strong. Color, immature: 178A. Color, mature: 137A.
Foliage description:
   Arrangement.—Alternate, simple.
   Length.—About 12 cm.
   Width.—About 10 cm.
   Shape.—Oblong to ovate.
   Apex.—Obtuse to acute.
   Base.—Cordate.
   Margin.—Serrate.
   Texture, upper and lower surfaces.—Slightly pubescent.
   Venation pattern.—Pinnate.
   Color.—Developing foliage, upper and lower surfaces: 187A. Fully expanded foliage, upper surface: Spring and summer, 183B; late summer and fall, 137A. Fully expanded foliage, lower surface: Spring and summer, 178A; late summer and fall, 137A. Venation, upper surface: Spring and summer, 183B; late summer and fall, 137A. Venation, lower surface: Spring and summer, 178A; late summer and fall, 138B.
   Petiole.—Length: About 1 cm. Diameter: About 2.5 mm. Texture, upper and lower surfaces: Pubescent. Color, upper surface: Spring and summer, 183B; late summer and fall, 137A. Color, lower surface: Spring and summer, 178A; late summer and fall, 138B
Flower description: Male inflorescences are catkins, color prior to elongation 176B. Female inflorescence style color 183B.
Disease/pathogen/pest resistance: Plants of the new Corylus are resistant to eastern filbert blight caused by the fungus Anisogramma anomala (Peck) E. Muller. Plants of the new Corylus are moderately susceptible to bud mites (Phytoptus avellanae Nal.) as are plants of ‘Contorta.’
Temperature tolerance: Plants of the new Corylus have been observed to tolerate temperatures from about -10° C. to about 38° C. in the field in Corvallis, Oreg.

Trees of ‘Red Dragon’ set a moderate number of catkins (rating=2.3) which is less than ‘Contorta’ (rating=3.2) but more than other contorted selections. The catkins elongate in late winter with ‘Contorta.’‘Red Dragon’ has incompatibility alleles S₆ and S₂₆ as determined by fluorescence microscopy. Both alleles are expressed in the females, but only S₆ is expressed in the pollen because of dominance. Female inflorescences of ‘Red Dragon’ also emerge late in the season, with ‘Contorta.’ ‘Red Dragon’ trees will set a few nuts if its stigmas receive compatible pollen while receptive. The nuts are small, slightly long and compressed. The nuts are borne in clusters of one or two in husks equal in length to the nuts. Pollen of the red-leaf cultivars ‘Rode Zeller’ and ‘Fusco Rubra’ expresses S₆ and, thus, is incompatible. ‘Contorta’ (S₅ S₁₀) is reciprocally compatible with ‘Red Dragon.’

DNA was extracted from several contorted seedlings and amplified by PCR. Random amplified polymorphic DNA (RAPD) markers UBC 152-800 and UBC 268-580, which flank the ‘Gasaway’ resistance gene, are present in ‘Red Dragon.’ RAPD marker AA12-850, which co-segregates with resistance, is also present. Scions were collected from ‘Red Dragon’ and several other contorted selections and three trees of each were grafted to rooted layers of Corylus avellana. The shoot tips of the grafted trees were inoculated in the greenhouse with a spore suspension of Anisogramma anomala and then held under high humidity. The three inoculated trees of ‘Red Dragon’ remained free of disease, while those of other selections in the same test developed cankers. The lack of cankers confirmed the results of the RAPD markers and indicates complete resistance to eastern filbert blight.

Notably, no trees have been lost to bacterial blight caused by Xanthomonas campestris pv. corylina, however susceptibility to the disease has not been rigorously tested.

Susceptibility to big bud mite (primarily Phytoptus avellanae Nal.) was rated after leaf fall once per year for three years. The scale was from 1 (no blasted buds) to 5 (many blasted buds). The average bud mite rating for ‘Red Dragon’ (3.2) is slightly higher than for ‘Contorta’ (2.6), but the difference is not significant at P=0.05. The nursery trade does not consider bud mite to be a serious problem for ‘Contorta.’ Therefore, bud mite should also not be a serious problem for ‘Red Dragon.’

Fingerprinting with simple sequence repeat (SSR) markers was also performed. A panel of simple sequence repeat marker loci for hazelnut has been developed. Using primers designed for each SSR locus, hazelnut DNA was amplified by PCR as described (Bassil et al. (2005) J. Amer. Soc. Hort. Sci., 130:543-549). The sequences of the primers are provided in Table 1. Forward primers were fluorescently labeled with FAM, HEX, or NED, and the size of the amplified fragment was determined by capillary electrophoresis on an ABI 3100 instrument (Applied Biosystems; Foster City, Calif.). 93 SSR loci were used to amplify 32 hazelnut genotypes. Microsatellite markers in hazelnut are described in Bassil et al. (J. Amer. Soc. Hort. Sci. (2005) 130:543-549), Bassil et al. (Acta Horticulturae (2005) 686:105-110), Boccacci et al. (Mol. Ecol. Notes (2005) 5:934-937), Boccacci et al. (Genome (2006) 49:598-611), and Mehlenbacher et al. (Genome (2005) 49:122-133). The disclosure of each of the above citations is incorporated by reference herein.

TABLE 1

Microsatellite markers used to fingerprint ‘Red Dragon’
and other hazelnuts.

| Locus | Forward Primer Sequence | Reverse Primer Sequence |
|---|---|---|
| A040 | TGCTCAAGCAAATATTGCAC | GTTTGGGATCCAATTAACCCTCT |
| AJ490 | ACTGCAGCCCTTCAACTACG | AACGCCTACCTCATGTTTGG |
| B107 | GTAGGTGCACTTGATGTGCTTTAC | AACACCATATTGAGTCTTTCAAAGC |
| B507 | CTAAGCTCACCAAGAGGAAGTTGAT | GCTTCTGGGTCTCCTGCTCA |
| B508 | GGTCAAGATTTGATAAAGTGGGA | GCACTCCACTTGTGCGTTTTC |
| B617 | TCCGTGTTGAGTATGGACGA | TGTTTTTGGTGGAGCGATG |
| B619 | AGTCGGCTCCCCTTTTCTC | GCGATCTGACCTCATTTTTG |
| B643 | CCAGGTAAGCAGCTCAGTGT | ACCTCCCATTTGTGTCTTGC |
| B662 | CGAAAGATGGACTTCCATGAC | CAAGTTGAGATTCTTCCTGCAA |
| B709 | CCAAGCACGAATGAACTCAA | GCGGGTTCTCGTTGTACACT |
| B720 | CTCTGTGTCGGCTTTCTGGT | ATAAACCTCACGCCACACCT |
| B732 | GCCCTTCTTCTTTTCTGCAA | AGTGCCACCTCAACAAATCC |
| B733 | CACCCTCTTCACCACCTCAT | CATCCCCTGTTGGAGTTTTC |
| B741 | GTTCACAGGCTGTTGGGTTT | CGTGTTGCTCATGTGTTGTG |
| B774 | GTTTTGCGAGCTCATTGTCA | TGTGTGTGGTCTGTAGGCACT |
| B776 | TGTATGTACACACGGAGAGAGAGA | TGAGGGGAAGAGGTTTGATG |
| C010 | GGAGCCACCATGAAATTATACA | CACTTATTGCGATTGGTTCA |
| C028 | CTACCCCATCGCTTGACAC | GGAGACTTGTTTGCCACAGA |
| C119 | CTCACCTTTACCCCTTCATTTT | GTTTCCTCATCTTCTGAGAACCATC |
| C504 | GGTCTCCTTCGCTAACATAACCAA | GTTGCCCTCGAGTTGTAGTA |
| K27-28-30 | ACACACACACACACACGAA | GCACCAAGCAGAACAATC |
| K74-1-19 | CTTACAGATAAATGGCTCAAA | AAGCAAGAAAGGGATGGT |
| K74-2-36 | AGGCATCAGTTCATCCAA | GGAAGGTGAGAGAAATCAAGT |
| K74-6-19 | TTATTCCACCAAAGTCTACCTC | TCCTCACCAATCACACTATTT |
| K76-1-26 | AAGGCGGCACTCGCTCAC | GAACAACTGAAGACAGCAAAG |
| K80-1-2 | CTGGCTTCAAATCAATCATAC | GAGGAGAGGAGAGAGAAAGAG |

| Locus | Repeat Motif | Size | Tm | SEQ ID NO (F, R) |
|---|---|---|---|---|
| A040 | (CA)₁₃ | 234-248 | 62 | 1, 2 |
| AJ490 | (CT)14Ns(CT)15 | 210-224 | 60 | 3, 4 |
| B107 | (CT)₁₄ | 112-151 | 58 | 5, 6 |
| B507 | (GA)1GC(GA)2GC(GA)14 | 182-202 | 55 | 7, 8 |
| B508 | (GA)₁₀ | 142-167 | 62 | 9, 10 |
| B617 | (GA)₁₅ | 280-298 | 60 | 11, 12 |
| B619 | (TC)₂₁ | 146-180 | 60 | 13, 14 |
| B643 | (AG)₁₃ | 180-196 | 60 | 15, 16 |
| B662 | (TC)₁₅ | 220-236 | 60 | 17, 18 |
| B709 | (GA)₂₁ | 219-233 | 60 | 19, 20 |
| B720 | (AG)₁₄ | 159-179 | 60 | 21, 22 |
| B732 | (GA)₁₃ | 140-156 | 60 | 23, 24 |
| B733 | (TC)₁₅ | 161-183 | 60 | 25, 26 |
| B741 | (GT)₅(GA)₁₂ | 176-194 | 60 | 27, 28 |
| B774 | (AG)₁₅ | 195-213 | 60 | 29, 30 |
| B776 | (GA)₁₇ | 134-148 | 60 | 31, 32 |
| C010 | (GAA)^{a} | 272-319 | 58 | 33, 34 |
| C028 | (GAA)₁₀ | 131-147 | 60 | 35, 36 |
| C119 | (GA)₇(GA)₉ | 256-264 | 62 | 37, 38 |
| C504 | (CT)18 | 161-187 | 55 | 39, 40 |
| K27-28-30 | (AG)₁₀ | 328-345 | 57 | 41, 42 |
| K74-1-19 | (AAAT)₅ | 255-249 | 54 | 43, 44 |
| K74-2-36 | (AGG)₇ | 328-346 | 55 | 45, 46 |
| K74-6-19 | (AG)₁₅ | 366-393 | 56 | 47, 48 |
| K76-1-26 | (GA)₁₇ | 240-278 | 58 | 49, 50 |
| K80-1-2 | (GA)₁₃ | 188-211 | 57 | 51, 52 |

Shown for each microsatellite marker locus are the sequence of the forward and reverse primers, the repeat motif, the range of sizes generated, the annealing temperature, and the sequence identifiers.
^{a}(GAA)₇GGA(GAA)₂N₂₁(GAA)₂ATT(GAA)₄N₁₅(GAA)₃.

The allele sizes at 26 loci that distinguish 12 hazelnut genotypes are presented below (Table 2). OSU 217.094 is a red leaf seedling of ‘Contorta,’ and its pollen parent is believed to be ‘Rode Zeller.’ DNA of six contorted red leaf selections (two selections of the ‘Red Majestic,’ ‘Red Dragon,’ OSU 897.046, OSU 897.071 and OSU 897.082) was also amplified. ‘Red Majestic’ plants from Spring Meadow and from Klehm are clearly different, as they have different alleles at 19 of the 26 loci. Notably, ‘Red Dragon’ is different from both clones of ‘Red Majestic’ and from all other genotypes in Table 2. Indeed, ‘Red Dragon’ was found to exhibit different allele sizes at certain loci that allowed for it to be distinguish from other hazelnut genotypes such as ‘Contorta’ and ‘Red Majestic.’

TABLE 2
Allele sizes at 26 microsatellite loci of 12 hazelnut genotypes.

| Genotype Locus | A040 | AJ490 | B107 | B507 | B508 |
|---|---|---|---|---|---|
| Gasaway | 238/246 | 218/222 | 122/128 | 179/190 | 147/165 |
| Rode Zeller | 238/246 | 214/216 | 134/144 | 189/197 | 149/149 |
| OSU 217.094 | 238/246 | 214/214 | 130/134 | 185/197 | 157/157 |
| Contorta | 246/246 | 212/214 | 122/130 | 185/189 | 147/155 |
| Red Majestic Spring | 246/246 | 212/212 | 122/130 | 189/189 | 147/157 |
| Meadow | | | | | |
| Red Majestic Klehm | 246/246 | 214/214 | 122/130 | 189/189 | 147/149 |
| OSU 897.046 | 238/246 | 212/214 | 128/134 | 179/197 | 147/157 |
| OSU 897.071 | 246/246 | 214/224 | 122/130 | 179/197 | 157/165 |
| Red Dragon (OSU | 246/246 | 212/216 | 128/130 | 185/189 | 165/165 |
| 897.078) | | | | | |
| OSU 897.082 | 246/246 | 212/216 | 122/134 | 179/185 | 165/165 |
| FuscoRubra | 238/248 | 214/222 | 120/122 | 189/199 | 165/165 |
| Barcelona | 236/236 | 212/224 | 112/134 | 181/191 | 157/157 |
| Genotype Locus | B617 | B619 | B643 | B662 | B709 |
| Gasaway | 292/296 | 170/174 | 190/196 | 230/236 | 227/227 |
| Rode Zeller | 282/292 | 166/176 | 192/192 | 230/230 | 227/227 |
| OSU 217.094 | 292/294 | 170/176 | 190/190 | 230/236 | 227/227 |
| Contorta | 282/294 | 156/170 | 182/192 | 232/236 | 221/227 |
| Red Majestic Spring | 282/282 | 156/170 | 192/192 | 226/232 | 227/227 |
| Meadow | | | | | |
| Red Majestic Klehm | 282/294 | 170/176 | 186/196 | 226/232 | 221/227 |
| OSU 897.046 | 294/294 | 156/176 | 182/192 | 230/230 | 227/227 |
| OSU 897.071 | 282/292 | 156/166 | 182/192 | 226/236 | 221/227 |
| Red Dragon (OSU | 282/292 | 156/156 | 182/192 | 226/236 | 225/227 |
| 897.078) | | | | | |
| OSU 897.082 | 282/292 | 156/156 | 190/196 | 232/236 | 221/227 |
| FuscoRubra | 290/296 | 156/166 | 182/192 | 222/228 | 225/227 |
| Barcelona | 286/290 | 156/170 | 190/190 | 230/232 | 225/233 |
| Genotype Locus | B720 | B732 | B733 | B741 | B774 |
| Gasaway | 161/163 | 140/154 | 173/173 | 186/188 | 203/209 |
| Rode Zeller | 159/167 | 150/150 | 173/173 | 178/184 | 203/207 |
| OSU 217.094 | 167/167 | 150/154 | 161/173 | 178/184 | 203/203 |
| Contorta | 159/167 | 142/154 | 161/161 | 163/184 | 203/203 |
| Red Majestic Spring | 167/167 | 142/142 | 161/161 | 178/184 | 203/203 |
| Meadow | | | | | |
| Red Majestic Klehm | 159/167 | 140/140 | 161/161 | 163/178 | 203/209 |
| OSU 897.046 | 163/167 | 140/154 | 161/161 | 178/186 | 203/209 |
| OSU 897.071 | 159/159 | 140/154 | 161/161 | 163/178 | 203/203 |
| Red Dragon (OSU | 163/167 | 140/150 | 161/161 | 178/178 | 203/209 |
| 897.078) | | | | | |
| OSU 897.082 | 159/167 | 140/150 | 161/161 | 163/178 | 203/203 |
| FuscoRubra | 165/171 | 146/150 | 173/183 | 178/186 | 197/213 |
| Barcelona | 161/167 | 150/154 | 171/173 | 177/186 | 203/207 |
| Genotype Locus | B776 | C101 | C028 | C119 | C504 |
| Gasaway | 144/148 | 281/281 | 144/144 | 258/264 | 161/164 |
| Rode Zeller | 134/136 | 275/278 | 132/141 | 256/264 | 158/164 |
| OSU 217.094 | 136/146 | 275/275 | 132/141 | 256/258 | 161/164 |
| Contorta | 134/146 | 281/281 | 132/132 | 258/264 | 158/161 |
| Red Majestic Spring | 134/146 | 275/275 | 132/141 | 258/264 | 158/161 |
| Meadow | | | | | |
| Red Majestic Klehm | 146/146 | 281/281 | 132/135 | 258/264 | 152/161 |
| OSU 897.046 | 136/148 | 275/275 | 141/144 | 256/258 | 161/161 |
| OSU 897.071 | 134/148 | 275/281 | 132/132 | 258/258 | 161/161 |
| Red Dragon (OSU | 134/148 | 275/275 | 132/141 | 256/258 | 161/161 |
| 897.078) | | | | | |
| OSU 897.082 | 146/148 | 275/281 | 132/141 | 258/258 | 161/161 |
| FuscoRubra | 134/136 | 278/278 | 135/135 | 256/264 | 152/164 |
| Barcelona | 134/136 | 275/278 | 132/141 | 258/258 | 155/155 |

| Genotype Locus | K27-28-30 | K74-1-19 | K74-2-36 |
|---|---|---|---|
| Gasaway | 331/333 | 239/248 | 337/346 |
| Rode Zeller | 329/331 | 234/234 | 340/343 |
| OSU 217.094 | 329/329 | 234/248 | 337/340 |
| Contorta | 331/331 | 239/248 | 337/337 |
| Red Majestic Spring | 329/331 | 239/248 | 337/337 |
| Meadow | | | |
| Red Majestic Klehm | 329/329 | 239/239 | 337/343 |
| OSU 897.046 | 331/331 | 234/248 | 337/340 |
| OSU 897.071 | 331/331 | 239/248 | 337/340 |
| Red Dragon (OSU | 331/331 | 234/248 | 337/340 |
| 897.078) | | | |
| OSU 897.082 | 331/331 | 234/248 | 337/337 |
| FuscoRubra | 329/329 | 248/248 | 337/346 |
| Barcelona | 329/331 | 234/248 | 337/337 |

| Genotype Locus | K74-6-19 | K76-1-26 | K80-1-2 |
|---|---|---|---|
| Gasaway | 378/390 | 255/259 | 194/197 |
| Rode Zeller | 392/392 | 253/255 | 201/201 |
| OSU 217.094 | 376/392 | 253/253 | 201/201 |
| Contorta | 376/376 | 253/255 | 201/203 |
| Red Majestic Spring | 376/392 | 253/255 | 203/203 |
| Meadow | | | |
| Red Majestic Klehm | 376/376 | 253/255 | 199/203 |
| OSU 897.046 | 376/390 | 253/253 | 197/201 |
| OSU 897.071 | 376/376 | 251/253 | 197/201 |
| Red Dragon (OSU | 376/392 | 253/253 | 201/203 |
| 897.078) | | | |
| OSU 897.082 | 376/390 | 253/253 | 197/201 |
| FuscoRubra | 380/392 | 255/265 | 197/207 |
| Barcelona | 374/378 | 259/265 | 203/205 |

What is claimed is:

1. A new and distinct Corylus plant named ‘Red Dragon’ as illustrated and described.
